(19) United States
(12) Patent Application Publication
(10) Pub. No.: US 2019/0231686 A1
(43) Pub. Date: Aug. 1, 2019

(54) METHODS FOR TREATING CYSTIC FIBROSIS AND OTHER DISEASES AFFECTING MUCOSAL SURFACES

(71) Applicant: Lauranell Harrison Burch, Durham, NC (US)

(72) Inventor: Lauranell Harrison Burch, Durham, NC (US)

(21) Appl. No.: 16/318,158

(22) PCT Filed: Jul. 15, 2017
(86) PCT No.: PCT/US17/42280

§ 371 Date: Jan. 16, 2019

Related U.S. Application Data
(60) Provisional application No. 62/363,225, filed on Jul. 16, 2016.

(51) Int. Cl. A61K 9/00; A61P 31/04; A61P 29/00

(52) U.S. Cl. CPC A61K 9/0078; A61P 29/00; A61P 31/04

(57) ABSTRACT

A method of treating and/or managing cystic fibrosis (CF) and/or other infectious or inflammatory lung disease or mucosal surface condition in a patient in need thereof, using a drug formulation with beneficial and/or synergistic effects among ingredients which comprise the formulation herein reported. Cystic fibrosis disease is characterized by loss of homeostatic balance at mucosal surfaces, resulting in persistent infection, excessive sticky mucus and tissue-destructive host inflammatory responses. When used to treat lung disease, this invention is delivered to airways as an inhaled dose by nebulizer. The formulation(s) arising from this invention are preferably composed of natural endogenous compounds already found within the body but possibly obtained from other sources, and/or of plant phytochemicals, which in combinations herein disclosed, have synergistic hydrating, anti-inflammatory and antimicrobial properties as well as direct corrector and/or potentiator effects and/or other modulating effects on CFTR function when administered to mucosal surface where CFTR with some residual function is achieved. Synthetic molecules can also be used.

### STATEMENT OF PRIORITY

This application claims the benefit, under 35 U.S.C, § 1 19(e), of U.S. Provisional Application Ser. No. 62/363,225, filed Jul. 16, 2016, the entire contents of which are incorporated by reference herein.

### FIELD

The present invention is a method of treating and/or managing cystic fibrosis and other inflammatory or obstructive lung diseases or disease of a mucosal surface in a patient by administering a formulation derived from this invention to the affected mucosal surface. The formulation is administered by nebulization in the case of lung disease. The treatment restores hydration, increases pH and antimicrobial defense while reducing inflammation, directly modulating CFTR to increase residual CFTR function, and enhancing ciliary activity of a ciliated mucosal surface. The invention is disclosed below, along with compounds and compositions useful for carrying out such methods.

### BACKGROUND

The term ‘mucosal’ refers to tissues that produce mucus; mucosal tissues protect surfaces of structures such as the oral and oropharyngeal cavities and lungs in addition to lining the lumen of conducting tubules throughout the body. Mucosal secretions deliver to mucosal surfaces a variety of soluble factors including macromolecules, small molecules and ions that work synergistically to create healthy homeostasis by limiting host exposure to pathogens and by minimizing inappropriate inflammatory responses. Properly functioning, molecules that bathe airway mucosal surfaces eliminate infectious challenges without need for a second line of defense. Although breathing continuously exposes the airways to potential pathogens, bacterial infection at the mucosal surfaces of conducting airways is rare with a notable exception; the chronic lung infections that are the hallmark of the fatal genetic disease cystic fibrosis. Cystic fibrosis (CF) is the most common fatal genetic disease among Caucasians. Although the clinical features of cystic fibrosis involve multiple organs, the primary cause of morbidity and mortality is chronic pulmonary infections. Cystic fibrosis (CF) is caused by mutations in a single gene: the cystic fibrosis transmembrane regulator (CFTR). CFTR controls transport of multiple ions responsible for proper hydration and the anti-inflammatory and antimicrobial defense of mucosal surfaces. Loss of CFTR function results in accumulation of viscous secretions and repeated lung infections. Simply stated, cystic fibrosis airways are highly susceptible to microbial infection and inflammation while non-CF airways are resistant. As CF is a disease resulting from mutations in a single gene, the dramatic CF vs non-CF represent loss of functions directly attributable to and controlled by CFTR.

Currently there is no effective drug to prevent disease progression for most CF mutations; standard therapy relies heavily on repeated use of antibiotics that ultimately fail to eradicate lung infection and lead to emergence of multi-drug resistant pathogens. Decline in lung function is seen even in early childhood, and leads to requirement for lung transplant or premature death by respiratory failure. Among substrates known to be dependent, entirely or in part, on CFTR for transport to mucosal surfaces are glutathione, bicarbonate and thiocyanate, all of which serve critical roles in airway defense against inflammation and infection. These findings of transport dependence of multiple large ions by CFTR should not be surprising, as cloning and sequencing of CFTR in 1989 revealed that CFTR is a member of the ABC transporter gene family. Genes of the ABC transporter family actively transport multi-atomic molecules across a membrane in one direction using the energy of ATP to do the ‘pumping’. It is essential to keep in mind that there are some CFTR mutations, called chloride-conducting mutants, which move chloride ions normally but still cause CF disease. There are at least sixteen such mutations. If patients can move chloride ions through CFTR normally and have severe and progressive CF lung disease, this must mean that transport of the multi-atomic substrates by CFTR (such as bicarbonate, glutathione, and thiocyanate), in other words the ABC transporter function of CFTR, is essential to defense of airways against infection.

### SUMMARY

The invention described here, when used in the proper composition and dose to treat affected mucosal surfaces, can restore hydration, some innate immune defenses against pathogens and to reduce inflammation that is initiated even prior to infection via basal aberrant cytokine release by CF airway epithelial cells. We propose that hypertonic solutions routinely used to treat CF patients via nebulization are direct irritants that injure airway epithelial cells, inducing release of cytokines and inflammation and are long known in the literature to reduce needed cilia function. Exposure of airway cells to hyperosmotic challenge causes a series of unfavorable mechanical and biochemical events. Water rapidly moves out of cells, causing cells to shrink. Hyperosmolarity triggers release of chemical mediators from epithelial cells including cytokines; and can also trigger histamine release from mast cells, which can provoke an asthmatic response in sensitive individuals with reactive airways. Patients with reactive airways represent a significant subset of the CF population. Therefore, osmolarity must be carefully optimized and component ingredients of this invention and other future therapies. Nebulized formulations must be well-chosen, such that every represented molecule contributing to osmolarity of the drug fulfils a role with sufficient but not excessive concentration and dose.

### DETAILED DESCRIPTION

The term “(NO)” as used herein means nitric oxide.

The term (NOS)” as used herein means nitric oxide synthase.

The term “(iNOS)” as used herein means inducible nitric oxide synthase.

The term “Amino acid” as used herein means any amino acid that is involved in the nitric oxide pathway or in the urea cycle and is intended to include formulation, formulation salt or formulation analog, ornithine, ornithine salt or analog, proline, proline salt or analog, glutamine, glutamine salt or analog, alanine, alanine salt or analog and arginine, arginine salt or arginine analog.

The term “(CF)” as used herein means cystic fibrosis.

The term “(PAH)” as used herein means pulmonary arterial hypertension.

The term “(IPF)” as used herein means idiopathic pulmonary fibrosis.

The term “(FPF)” as used herein means familial pulmonary fibrosis.

The term “(ARDS)” as used herein means acute respiratory disorder syndrome.

The term “(PPHN)” as used herein means persistent pulmonary hypertension of the newborn.

The term “(PCD)” as used herein means primary ciliary dyskinesia.

The term “(COPD)” as used herein means chronic obstructive pulmonary disease.

The term “(ALI)” as used herein means acute lung injury.

The term “(FEV₁)” as used herein means forced expiratory volume defined as the maximum amount of air expired in one second.

The term “(MMAD)” as used herein means mass median aerodynamic diameter.

The term “substantially” as used herein means at least 70% and up to 90%.

The term “predominantly” as used herein means at least 90% and above.

The term “modulate CFTR function” as used herein means to increase CFTR activity and/or stability and/or messenger RNA level.

The term “active component” as used herein means formulation ingredient with a beneficial drug action and their salts or analogs.

As used throughout, ranges provided incorporate each and every value that is within the range. Any value within the range can be selected as the terminus of the range.

The term “airway surface” as used refers to airway surfaces below the larynx and in the lungs, as well as air passages in the head, including the sinuses, in the region above the larynx.

Unless otherwise specified, all percentages and amounts expressed herein and elsewhere in the specification should be understood to refer to percentages by weight. The amounts given are based on the weight of the material. The recitation of a specific value herein is intended to denote that value, plus or minus a degree of variability to account for errors in measurements. For example, an amount of 10% can include 9.5% or 10.5%, given the degree of error in measurement that will be appreciated and understood by those having ordinary skill in the art.

An “effective amount” as used herein, means an amount of active ingredient sufficient to produce a selected effect.

As used herein, “antibacterial activity” means activity to limit bacterial growth as determined by any generally accepted in vitro or in vivo antibacterial assay or test.

“Anti-inflammatory activity” herein means activity as determined by any generally accepted in vitro or in vivo assay or test, for example an assay or test for any marker of inflammation, such as production of cytokines, prostaglandins or 8-isoprostane.

“Antioxidant activity” herein means activity as determined by any generally accepted in vitro or in vivo antioxidant assay or test.

The expression “natural extract” as used herein denotes any extract that is obtained from a natural source, such as a plant, fruit, root, tree, and the like. The expression “natural compound” as used herein denotes any individual molecule isolated from a plant or natural extract.

Classification herein of an ingredient as an active agent or a buffering agent or preservative is made for clarity and convenience, and no inference should be drawn that a particular ingredient necessarily functions in the composition in accordance with its classification herein.

Furthermore, a particular ingredient in this invention can serve a plurality of functions, thus disclosure of an ingredient herein as exemplifying one function or functional class does not exclude the possibility that it can also exemplify another function or functional class.

As people with normal CFTR are resistant to infection of the conducting airways, and patients with mutant CFTR are extremely susceptible, restoring these multi-atomic substrates transported by normal CFTR to the airway surface through nebulization would be predicted to support antimicrobial defense of CF lungs, and some CFTR-transported substrates are incorporated in the invention disclosed here.

CF airways are not equally permissive to growth of all pathogens. Characteristically, Staphylococcus aureus is an early CF airways colonizer followed by establishment of chronic lung infection with highly adaptive Pseudomonas aeruginosa. Anions transported by CFTR to airway surface liquid act as critical cofactors for several innate antimicrobial defense factors—and prevent inappropriate, host-destructive inflammatory responses initiated through the airway epithelial surfaces. Failure of defective CFTR to transport these defense factors create conditions favorable to exploitation by specific pathogens. The adaptations of P. aeruginosa that ultimately lead to morbidity and mortality of the CF patient are associated with an exaggerated inflammatory response that is characterized by massive numbers of activated neutrophils in the conducting airways, producing both reactive oxygen and reactive nitrogen species. These features suggest that the airway environment resulting from the loss of CFTR function leads to both a reduction in the antimicrobial properties of the airway surface liquid and to pro-inflammatory hyper-responsiveness of the airway epithelial cells. In response to the CF airway environment, P. aeruginosa adapts by becoming hyper-mutable, favoring biofilm formation and development of antibiotic resistances. These events present special challenges for the formulation of potential therapeutic interventions. A successful treatment must interrupt the inflammation, mucosal surface dehydration, infection and pathologic progression characteristic of cystic fibrosis morbidity and mortality. Ideally, a well formulated combination drug therapy, such as the invention reported here, will remove selective advantages exploited by pathogens to colonize CF lungs, while preserving an appropriate environment for commensal (friendly) bacteria that are part of the first line of defense against pathogens via competition.

Lactoferrin (LF), lactoperoxidase (LPO), lysozyme (LZ), secretory IgA (S-IgA) and mucins are among the principal antimicrobial proteins found in mucosal secretions of the conducting airways, sinuses and the oral cavity. Function of both LF and LPO are dependent on molecules delivered to thbe airway surface liquid (ASL) by CFTR. Hydrogen peroxide that is required by LPO is synthesized by the NADPH oxidase dual oxidase 2 (DUOX 2), which is expressed by ductal epithelial cells. Hydrogen peroxide is also produced by inflammatory cells in the CF conducting airways. Functional CFTR is essential to transport of several anions including bicarbonate (HCO₃⁻), reduced glutathione (GSH) and contributes (along with pendrin) to the transport of thiocyanate (SCN⁻). All of these molecules have known critical roles in host mucosal surface bacterial defense. Various exocrine secretions of patients with CF are deficient in GSH, HCO₃⁻ SCN⁻, NO and also ascorbate, which is commonly depleted by infection. LPO catalyzes oxidation of SCN⁻ by H₂O₂ to form hypothiocyanate (OSCN⁻), a potent antimicrobial species that works by oxidizing essential sulfhydryl's of target proteins on bacterial surfaces. This oxidation can effectively inhibit bacterial metabolism through neutralization of enzymes such as hexose kinases required by bacteria for transport of sugars. Unlike its neutrophil counterpart myeloperoxidase (MPO), LPO cannot use the halide Cl⁻ as a substrate and will not generate the potentially host-noxious product hypochlorous acid, HOCl, (commonly known as bleach). While having potent antimicrobial activity, OSCN⁻ has the added advantage that it is relatively innocuous to host tissues. Therefore in the presence of an adequate concentration of SCN⁻ and functioning LPO, there would be competition for H₂O₂ that would limit activity of MPO's generation of HOCl and favor formation of OSCN⁻. There is also evidence that MPO can use SCN⁻ in preference to Cl⁻. Conversely, limitations in SCN⁻ due to defective CFTR transport and/or diet would serve to favor the less discriminating and more reactive HOCl. Furthermore, SCN reacts non-enzymatically with HOCl, converting it to the more microbe-specific OSCN. Therefore, in the presence of proper amounts of thiocyanate in ASL, production of tissue damaging hypochlorous acid is reduced. This may explain, at least in part, why higher thiocyanate levels are associated with better lung function in CF patients.

CF sweat, saliva, tears, nasal secretions and airway surface liquid, though depleted overall in other osmolytes, are nonetheless overly sodium chloride salty. In the presence of excess chloride in the CF lung, production of excess tissue-damaging hypochlorous acid in the airways would be predicted, as discussed above. As CF lungs are characterized by massive neutrophil infiltration in the conducting airways and abundant production of neutrophil products such as MPO, treatment of lungs with nebulized solutions containing additional chloride ions, such as hypertonic saline, would be predicted to exacerbate the problem of excessive production of hypochlorous acid and tissue damage. Injured cells release additional mediators of inflammation which would perpetuate the inflammatory cycle. Therefore treatment of CF patients via nebulization of molecules that liberate chloride ions is avoided in this invention. Lactoferrin (LF) has ability of high-affinity binding of two ferric ions in coordination with the binding of carbonate. This synergistic binding results in unusually high affinity; resulting in stabilization of iron in the ferric transition state. Other anions (e.g. HCO₃⁻) are also capable of fitting in to the anion coordinate site of LF, but can only occupy one of the two free coordinate sites. The remaining free coordinate site can then participate in a Haber-Weiss interaction in the presence of H₂O₂, generating a hydroxyl radical. This reaction could be amplified if there is sufficient reducing potential such as O₂⁻ or ascorbate supplied by this invention, which is available to cycle iron to a ferrous state. In addition to ascorbate's reducing potential, it has a size and configuration that it fits in the anion coordinate sites of lactoferrin, limiting stabilized carbonate-Fe³⁺ binding. If generated proximal to a susceptible bacterial surface, it would kill potential pathogens; conversely it could also damage host tissues. Therefore it would be predicted that iron binding by LF in coordination with carbonate would scavenge iron and serve an antioxidant function. In contrast, bicarbonate and ascorbate would serve to generate reactive oxygen species and bactericidal activity on the target pathogen surface. Therefore the ratio of carbonate to bicarbonate especially in the presence of other species such as ascorbate can serve an important regulatory function in determining antibacterial vs. antioxidant effects of LF. It is predicted that failure of CFTR to transport bicarbonate impairs antibacterial function of LF.

Ciliated epithelial cells express an inducible nitric oxide synthase, iNOS, which contributes to production of nitric oxide (NO⁻). Inducible NOS associated with phagocytic cells serves an antimicrobial function through the generation of NO that reacts with O₂⁻(also generated by phagocytic cells) to yield the highly reactive species peroxynitrite (ONOO⁻). Ideally this reaction occurs within phagosomes at the target pathogen surface and not extracellularly near mucosal surfaces where peroxynitrite could damage host tissues or otherwise lose effectiveness against target pathogens. In contrast, NO generated by the iNOS of ciliated epithelial cells is released to the airway surface environment; but in the normal individual, NO is generated in the presence of CFTR-transported glutathione. This NO reacts directly with this glutathione to form the S-nitrosothiol, nitrosoglutathione (GSNO), which is an important biologically active antimicrobial species at the airway surface. Our data indicate that GSH limits the ability of P. aeruginosa to utilize nitrate, nitrite or nitric oxide for respiration to grow under low oxygen conditions such as those encountered within biofilm in the CF lung. Furthermore nitrite and nitric oxide in the presence of GSH inhibit aerobic growth of P. aeruginosa, presumably via formation of the antimicrobial species GSNO. In the CF lung, failure of CFTR to release GSH at the airway surface prevents formation of sufficient antimicrobial GSNO, which results in unreacted NO (NO is also abundantly generated by neutrophils) which is then available for exploitation by nitrogen respiring pathogens for adaptation to colonization of the lung within biofilms.

Pseudomonas aeruginosa is adapted to gain selective advantage in the breakdown of effectiveness of innate mucosal surface defenses. In the CFTR-dysfunctional airway, there is striking predisposition to infection with P. aeruginosa, which ultimately transitions to the mucoidy phenotype associated with morbidity. It is reiterated here that reduced glutathione (GSH), which is transported to the airway surface by functional CFTR, serves to limit availability of nitrate necessary for pathogen growth in the oxygen-limited biofilm environment and GSH is necessary to promote antimicrobial activity of nitric oxide (NO) derived species. Furthermore, nitrosoglutathione (GSNO) delivered by normal, healthy airway epithelium serves to down-regulate recruitment of neutrophils, limiting their contribution to the cycle of host-destructive inflammatory processes.

The invention proposed here is a treatment formulated in part with glutathione or another thiol such as taurine, to restore formation of the antimicrobial species GSNO, or another nitrosothiol. Formation of a nitrosothiol (e.g. GSNO from glutathione and NO) also deprives nitrogen-respiring pathogens of a nitrogen source for respiration in biofilm. Reduced glutathione is also an important antioxidant, which provides in addition to its antimicrobial effects, some general protection against oxidative stress. In normal individuals, airway epithelial cells express iNOS to generate NO, which then acts in a negative feedback loop to terminate neutrophil migration into the airways. CF airway epithelial cells are deficient in the expression of iNOS compared to non-CF airways, as the expression of iNOS is mediated by reduced glutathione (GSH) and oxidized glutathione (GSSG). Because transport of GSH to mucosal surfaces is dependent on functional CFTR, CF patients lack GSH or GSSG in their ASL, resulting in down-regulated iNOS expression. Thus, there is a contribution to chronic neutrophil recruitment and sequestration within the CF conducting airways. When sufficient exogenous reducing equivalents are restored in the airways, as would be provided via glutathione and ascorbate administration and other reducing agents provided in this invention, then airway epithelial iNOS expression should increase. Inhaled L-arginine, an NO precursor, can be considered as a substrate to restore nitric oxide production for patients with cystic fibrosis. Am. J. Respir. Grit. Care Med., 174:208-212 (2006) reports pulmonary function improvement patients with cystic fibrosis with inhaled L-arginine. A single inhalation of L-arginine significantly increased exhaled NO, and also resulted in a sustained improvement of FEV₁. However, this treatment in the absence of co-administered GSH or other suitable thiol(s) would be predicted to be less efficacious due to deficiency of GSH to form nitrosoglutathione, and may serve the negative purpose in the absence of GSH of contributing to nitrogen respiration by colonizing pathogens. Control of inflammation and infection at mucosal surfaces is dependent on coordinated action of multiple proteins, molecules and ions and replacement of a subset of needed factors, or replacement in improper ratios and concentrations can exacerbate rather than correct the disease process. For example, supplying GSH and bicarbonate in the absence of other factors can be demonstrated in our laboratory to enhance pathogen growth.

Our recent data indicate that elevated CO₂ concentrations (as would be expected in the CF airway) resulted in dramatically enhanced expression of alginate by mucoid CF clinical isolates of P. aeruginosa. Furthermore, alginate expression was suppressed by titration with bicarbonate, which is deficient in CF airway surface liquid, as bicarbonate is transported by CFTR. It is our frequent observation in the laboratory that mucoid Pseudomonas clinical isolates can rapidly elaborate wildly abundant alginate biofilms, all arising from colony forming units (CFUs) so low in bacterial density that recovery of viable bacteria is difficult. Such alginate production in situ would likely be more than sufficient to occlude multiple small airways and physically block access of these regions to any nebulized drug. The exaggerated inflammatory response associated with the CF airway serves to provide an environment that selects for these unique adaptive properties of the CF pathogens. The invention disclosed here strategically intercedes in selective pressures that favor pathogens. We propose that approaches to treat CF will not be effective unless the inappropriate host inflammatory responses that lead to micro niche adaptations so characteristic of the successful CF pathogen are dampened and possibly redirected to antimicrobial function.

Both in vitro and in vivo studies have shown an up-regulation of inflammatory markers in CF, apparently with or without infection. This increased inflammatory signaling is a direct result of failure of CFTR transported substrates to modulate the airway innate immune response. This defect will be corrected by several complementary mechanisms by the proposed invention. An increase in pro-inflammatory cytokines and a decrease in the anti-inflammatory cytokine IL-10 could contribute to the characteristic influx of neutrophils to the airway lumen in the CF respiratory tract. Neutrophils would perpetuate the inflammatory cycle by participation in further recruitment and activation of more neutrophils through the release of leukotrienes and cytokines, as well as through proteolytic cleavage of structural and regulatory proteins such as complement and clotting cascades.

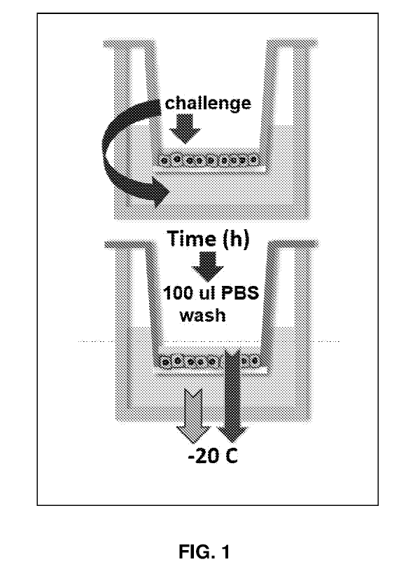

To determine CF vs non-CF differences in airway epithelial cell inflammatory signaling, we developed a reproducible cell culture model that can be used to measure the relative ability of drug treatments to control inflammation at the airway surface (FIG. 1). FIG. 1 describes the cell culture method to determine response to challenge agents and treatments. CF and non-CF control primary cultures or immortalized airway epithelial cells are grown to confluence on permeable membrane supports under air-liquid interface conditions and then are challenged/treated on the mucosal or serosal side. Mucosal challenge models an airway surface delivery (nebulization) and mucosal challenger models delivery of a test agent through the bloodstream. Mucosal surface wash and serosal media sample are collected for measurement of CFTR-transported substrates, for total metabolomics evaluation, and for cytokines/other mediators of inflammation by bead-based multiplex ELISA.

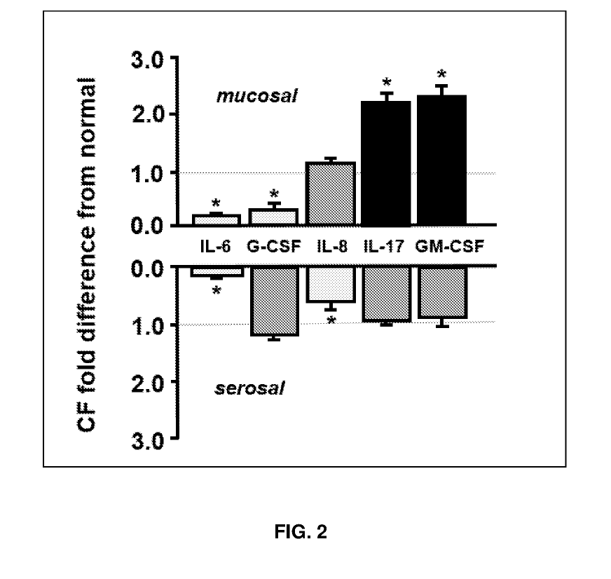

Neutrophil regulators were found to be increased under basal conditions (FIG. 2) and with inflammation challenge (IL-1B) in this model, but only significantly increased from the mucosal side. FIG. 2 describes polarity of basal cytokine release in CF vs normal primary epithelial cell cells grown in air-liquid interface. Light gray boxes represent significantly lower and black boxes significantly higher cytokine (P<0.05; N=3-5). In this initial study, we determined bilateral release of 17 cytokines in CF and normal nasal vs bronchial air-liquid interface primary cultures under basal conditions. Significant bilateral release was detected for all cytokines, and their expression was confirmed at the mRNA level. Bilateral basal release was lower overall in CF with the exception of neutrophil regulators IL-17 and GM-CSF at the mucosal side, which were both significantly increased, and IL-8 which trended toward increase but did not reach significance. Normal and CF nasal cultures were then compared for their ability to generate an inflammatory response to a mucosal surface challenge of 1 ng/ml IL-1B. After 24 hour exposure to this IL-1B challenge, mucosal release of IL-4, IL-6, IL-8, IL-10, IL-13 IL-17, IFNg and TNFa were all significantly increased in CF vs normal epithelia. Serosal cytokine release from normal and CF epithelia was not significantly affected by mucosal IL-1B. These results demonstrate fundamental differences in polarity of release of multiple cytokines in CF, and can in part explain the observed disconnect between increased inflammation in CF airways in vivo and inconsistent cytokine release reported in cell culture models, as most models fail to consider the potential bilateral nature of cytokine release by polarized epithelial cells. We find that neutrophil regulators are released in increased amount at the CF mucosal surface even under basal conditions, and restoration of specific molecules as specified in this invention restore normal mucosal surface properties such as cytokine signaling, airway surface hydration and ability to control bacterial infection at the mucosal surface.

Serosal cytokine release from normal and CF epithelia was not significantly affected by mucosal IL-1B. These results demonstrate fundamental differences in polarity of release of multiple cytokines in CF, and can in part explain the observed disconnect between increased inflammation in CF airways in vivo and inconsistent cytokine release reported in cell culture models, as most models fail to consider the potential bilateral nature of cytokine release by polarized epithelial cells.

This invention reflects the fact that mechanisms that control inflammation and infection at mucosal surfaces are complex and interdependent, and involve multiple interactions between host and pathogen. For example, in response to infection, incoming airways neutrophils produce NO, which is converted by CF pathogens to nitrate so that they can exploit the low oxygen environment of biofilm and survive under conditions of nitrogen respiration. CF patients colonized with pathogens capable of nitrate respiration likely show increased nitrate in their saliva for this reason. In the presence of nitrate, a treatment including glutathione and ascorbate, which are both deficient in CF airways, work synergistically to limit P. aeruginosa growth. This same treatment would spare commensal organisms as they are not producing nitrate. This invention, which concurrently addresses this mechanism, and also addresses multiple additional failures in innate immune defense will limit pathogens more effectively than any treatment previously known to the art. CFTR transports multiple substrates with important functions at mucosal surfaces, and restoring more of these functions will achieve greater therapeutic efficacy.

Therefore, this invention is a combination formulation comprised of ingredient components that: (1) restore molecules that defective CFTR fails to transport or molecules that are decreased in abundance in CF secretions, (2) restore extended hydration to the mucosal surface, (3) help to balance mucosal surface pH, (4) inhibit pathogen growth, (5) reduce markers of inflammation (6) directly or indirectly modulate CFTR function, (7) act as a mucolytic/dissolve biofilm and (8) increase function of cilia.

This invention can be prepared in a nebulized formulation of sufficient efficacy and duration of beneficial effects between treatments that it can be administered in some patients only morning and evening, eliminating a mid-day treatment burden that presents a lifestyle issue for CF patients. The invention described here can be used to prevent deterioration of lung function, development of bronchiectasis, cough, dyspnea, chronic airways infection, and ultimately respiratory failure in cystic fibrosis patients. In addition to treatment of cystic fibrosis, the invention can be used to treat other mucosal surface disease such as but not limited to: asthma, chronic bronchitis, non-CF bronchiectasis, pulmonary arterial hypertension (PAH), familial pulmonary fibrosis (FPF), idiopathic pulmonary fibrosis (IPF), acute respiratory distress syndrome (ARDS), persistent pulmonary hypertension of the newborn (PPHN), primary ciliary dyskinesia (PCD), chronic obstructive pulmonary disease (COPD), acute lung injury (ALI), and sarcoidosis. The invention can also be used to treat military wounds and burn wounds which are susceptible to biofilm growth by the same organisms that colonize CF lungs.

Other mucosal surfaces in need of treatment can also be the oropharyngeal cavities, nasal cavities, eyes, vulva, vagina and intestinal tract.

The present invention provides a method of treating and/or managing inflammatory and/or infectious diseases affecting mucosal surfaces by administering a combination drug therapy as an aqueous solution or in powder form directly to the affected mucosal surface(s) of the patient. If the condition to be treated is a lung disease, the invention can be administered by nebulization of the liquid or dry powder formulation. By “treating” or “managing” it is meant improving, preventing worsening of, and/or alleviating symptoms of a mucosal surface disease such as but not limited to: cystic fibrosis (CF) , asthma, chronic bronchitis, non-CF bronchiectasis, pulmonary arterial hypertension (PAH), familial pulmonary fibrosis (FPF), idiopathic pulmonary fibrosis (IPF), acute respiratory distress syndrome (ARDS), persistent pulmonary hypertension of the newborn (PPHN), primary ciliary dyskinesia (PCD), chronic obstructive pulmonary disease (COPD), acute lung injury (ALI), and sarcoidosis. In particular, treating cystic fibrosis includes causing one or more of the following: increasing FEV1, increasing blood oxygen saturation, enhanced CFTR activity, augmented airway hydration, raising airway surface liquid pH, reducing inflammation in the airways, improved mucociliary clearance, bronchodilation, and antimicrobial effects. Other mucosal surfaces in need of treatment can also be the oropharyngeal cavities, nasal cavities, eyes, vulva, vagina and intestinal tract. Patients with burn wounds and other injuries which can form biofilm infections such as military injuries can be treated with the present invention as such wounds are susceptible to biofilm growth by the same organisms that colonize CF lungs.

Subjects that can be treated by the method of the present invention also include patients on supplemental oxygen (which tends to dry airway surfaces), patients with an allergic disease or response (e.g., an allergic response to pollen, dust, animal hair or dander particles, insects or insect particles, or any other allergen) that affects airway surfaces, patients afflicted with a bacterial infections at a mucosal surface (e.g., Staphylococcus infections such as Staphylococcus aureus infections, Haemophilus influenza infections, Streptococcus pneumoniae infections, Pseudomonas infections, etc.), or patients afflicted with sinusitis (wherein the active agent or agents are administered to promote proper hydration and antimicrobial and/or anti-inflammatory defense of the sinuses).

The present invention can be used to hydrate and defend against inflammation and infection at mucosal surfaces other than airway surfaces. Such other mucosal surfaces include gastrointestinal surfaces, oral surfaces, genitourinary surfaces, ocular surfaces or surfaces of the eye, the inner ear, and the middle ear. For example, the active compounds of the present invention can be administered by any suitable means, including orally or rectally or vaginally. This invention can be used in the treatment of smoker's cough, inflammatory lung disease, pulmonary fibrosis, pulmonary vasculitis, pulmonary sarcoidosis, inflammation and/or infection associated with lung transplantation, acute lung rejection, burn wounds, chemical injury, military wound, pulmonary artery hypertension, bronchitis, sinusitis, asthma, ocular inflammation, ocular infection, dry eye, cystic fibrosis, bacterial infection, fungal infection, parasite infection, viral infection, chronic obstructive pulmonary disease (COPD), sinus infection, bronchiolitis obliterans syndrome (BOS), primary ciliary dyskinesia (PCD), alveolar proteinosis, idiopathic pulmonary fibrosis, familial pulmonary fibrosis, military wounds, burn wounds, eosinophilic pneumonia, eosinophilic bronchitis, acute lung injury, acute respiratory distress syndrome (ARDS), inflammation and/or infection associated with mechanical ventilation, ventilator-associated pneumonia, asbestos-related airway disorder or disease, dust-related airway disorder or disease, silicosis, chemical agent-related airway disease or disorder and any combination thereof.

The present invention is primarily concerned with treatment of human subjects, but can also be employed for treatment of other mammals for veterinary purposes.

The first aspect of the present invention is a method to supply the substrates which are normally transported by functional CFTR or a functionally equivalent molecule to the normally transported substrate; to a mucosal surface in need of such treatment. The method comprises the topical application to the mucosal surface of CFTR substrate molecules or functionally equivalent molecules, such as pharmaceutically acceptable salt salts of bicarbonate, reduced glutathione, pharmaceutically acceptable salt salts of thiocyanate, and any and all other pharmaceutically acceptable salt salts of molecules demonstrated to be transported by CFTR (or their functional equivalents), or shown to be differentially abundant in CF vs Non-CF secretions. Other molecules that are included in this invention can include plant alkaloids such as theophylline or theobromine among others, and/or dietary polyphenols such as ferulic acid, chlorogenic acid and vanillin, which can have anti-inflammatory and/or bronchodilation and/or hydrating (osmolyte) and/or antimicrobial effects. If the mucosal surface in need of treatment is an airway surface, the treatment can be applied through nebulization of an aerosolized liquid or powder drug formulation.

The second aspect of the invention provides a method for restoring extended hydration to the airways. CF airways have insufficient hydration to support proper mucociliary clearance. When hypertonic saline is nebulized into airways, there is an immediate shrinkage of cells and release of water to the airway surface. Then the airway surface liquid volume returns to baseline in minutes. Since airway surface liquid depth is depleted in CF, and depleted airway surface liquid volume is believed to contribute to impaired mucociliary clearance of the CF lung, molecules with more extended residence time at the mucosal surface that can restore hydration for longer periods of time are desirable. In contrast to sodium chloride, alcohol sugars Mannitol and Xylitol are examples of such molecules that can be used in this invention, with xylitol preferred over mannitol as xylitol is less likely than Mannitol to be utilized as a carbon source by bacteria. Sugar alcohols have long residence time at the mucosal surface, drawing water to hydrate the mucosal surface for a more extended period of time than sodium chloride. Other sugar alcohols that can be used include but are not limited to: sorbitol, maltitol, lactitol, and erythritol. The polysaccharide hyaluronic acid (HA) can be used in addition to an alcohol sugar to provide improved and prolonged water homeostasis/hydration of airway surfaces, and to provide other beneficial effects as are previously reported for this important molecule.

A third aspect of the invention is the upward adjustment of mucosal surface pH. Failure of defective CFTR to transport bicarbonate results in an airway surface liquid pH that is lower in CF than in normal individuals. This abnormal pH can be associated with reduced function of innate immune defense peptides and innate immune defense proteins in the lung. To restore a more favorable higher pH, this invention will incorporate a pharmacologically acceptable bicarbonate salt and/or also can include other buffering molecules including Tris (THAM), alkaline glycine buffer and phosphate buffers.

The fourth aspect of this invention incorporates methods to directly inhibit pathogen growth. Many plant extracts from plants that are used as foods, edible flavoring and/or aromatic herbs are known in the art to inhibit microbial growth while exhibiting superior safety profiles, as some of these plants and their extracts through historic use as foods qualify as GRAS (generally recognized as safe) ingredients by the US Food and Drug Administration (FDA). While not wishing to limit this invention by mechanism, certain plant compounds can interfere with bacterial growth by inhibition of quorum sensing by bacteria. Such plant-derived antimicrobial molecules that can be incorporated in the invention include, but are not limited to: carvarcrol, rosmarinic acid, thymol, estragol, allicin, menthol, reserpine, eugenol, anthemic acid, gallic acid, caffeic acid, linalool and p-cymene. Incorporation of such an antimicrobial plant molecule can serve to maintain a product manufactured from this invention free of microbial contamination (serving a preservative function) and can also serve the dual purpose of contributing to the antimicrobial efficacy of the administered therapy.

The fifth aspect of the invention is use of compound(s) that reduce inflammation at the mucosal surface. Compounds can have direct antioxidant effects and/or work indirectly to reduce production of pro-inflammatory cytokines and/or other markers of inflammation. Certain plant polyphenols are known to reduce inflammation in the body and can serve to reduce inflammation when incorporated in this invention. One example is green tea extract, which contains multiple beneficial molecules of which epigallocatechin gallate (EGCG) is an example. Green tea extract is an herbal derivative from the leaves of Camellia sinensis. Pycnogenol is a plant nutraceutical used since ancient times. Pycnogenol is derived from bark of the maritime pine tree (Pinus maritima) and its medicinal uses date back at least 2000 years. Pycnogenol is considered beneficial for wound healing and for reducing vascular inflammation. Pinus maritima bark extract contains active polyphenols including catechins, taxifolin, procyanidins, and phenolic acids. Pycnogenol inhibits TNFα-induced NF-KB activation, in addition to adhesion molecule expression in the endothelium; which can serve to limit neutrophil migration into airways in cystic fibrosis. Pycnogenol also statistically significantly inhibited the expression of inflammatory marker matrix metalloproteinase 9 (MMP-9). MMP-9 is highly expressed at sites of inflammation and contributes to pathogenesis of various chronic lung diseases. Other natural plant sources with extract compounds that can be used include Guggul, Holy basil, Neem, Boswellia serrata, Matricaria recutita (German Chamomile) Withania somnifera (ashwagandha), Zingiber officinale (ginger), and Curcuma longa [turmeric]. Alternatively, ibuprofen can be used. The amino acid taurine is a natural thiol antioxidant that is safely inhaled by nebulization. Taurine can serve in the fifth aspect of this invention to reduce inflammation at a mucosal surface. Taurine can also reduce inflammation by forming microbiocidal chlorine compounds that are less tissue damaging than HOCI. For example, N-chlorotaurine is the N-chloro derivative of the amino acid taurine. Astaxanthin is a carotenoid that gives carrots, salmon, shrimp and lobsters their orange color. Astaxanthin is a powerful antioxidant and can be used to reduce inflammation at the mucosal surface in this invention. Alternatively, n-acetyl cysteine or n-acetyl lysine can be used. Other aspects of this invention can contribute by various direct or indirect means to overall reduction of inflammation at the affected mucosal surface.

The sixth aspect of the invention comprises the method of topically applying molecules that directly modify CFTR function by correction and/or potentiation or other modulation of CFTR to enhance CFTR activity at the mucosal surface. Defective or insufficient CFTR can be corrected (restored to the cell surface) or potentiated (increased in activity) by a variety of polyphenolic molecules that are natural extracts and some that are FDA approved flavorings for human use. Alternatively, CFTR can be modulated in other ways, for example by increasing cyclic AMP within cells using compounds such as forskolin. Synthetic molecules can be used for CFTR modulation. However, plant-derived polyphenolic compounds are the preferred CFTR modulating molecules. Molecules are provided to the mucosal surface in an amount and combination effective to cause modulation of CFTR activity at that surface. Combinations of several correctors and/or potentiators and/or modulators are more efficacious than any single molecule to restore CFTR function. Polyphenolic molecules such as flavones and isoflavones, flavonoids, xanthines, terpenes, pentacyclic triterpenes, stilbenes and benzimidazoles are among the preferred classes of molecules that can be used. Curcuminoids, resveratrol, apigenin, naringen, luteolin and quercetin are examples of polyphenolic molecules that can be used in this invention as modulators of CFTR function. Some molecules can serve both to correct and potentiate CFTR.

A seventh aspect of this invention involves methods to thin mucus at a mucosal surface. This invention supplies molecules that are effective in thinning mucus. Molecules with sulfhydryl groups can be used in this aspect of the invention as molecules with sulfhydryl groups can directly attack disulfide bonds of mucins. Taurine is an amino acid that is demonstrated safe for inhalation and can serve this mucolytic role through the action of its sulfhydryl groups to break disulfide bonds of mucins. Guaifenesin, the natural plant molecule often used in popular over the counter expectorants can be used. Lumbrokinase, Nattokinase and Serrapeptase can also be used. Bicarbonate salts can also serve to thin mucus.

An eighth aspect of the invention involves methods to enhance the activity of cilia on a mucosal surface that is ciliated. This aspect is critical to treatment of respiratory diseases as good airway ciliary function serves to clear the airways of infection and debris. This invention utilizes molecules that are designed to perform various functions to restore proper hydration and anti-inflammatory and antimicrobial defenses to mucosal surfaces in need of such treatment. For most applications of this invention, the treatment formulation will be slightly hypertonic. However, excessively hypertonic solutions impair ciliary beat and will not be used. Excessively hypertonic solutions damage cilia. Osmolarity of treatments from this invention will be maintained between 250 and 1200 mOsM unless the invention is prepared specifically for applications where higher osmolarity can be required (such as burn wounds). Selection of pharmacologically acceptable salts is also critical to this invention with regard to ciliary beat. As secretions of the cystic fibrosis patient are already overly sodium chloride salty, use of sodium and chloride salts of active and inactive ingredients will be minimized or avoided altogether if possible. Sodium ions will be avoided because they decrease ciliary beat. Citrate ions will be avoided because they decrease ciliary beat. Magnesium is an important ion for increasing and maintaining ciliary activity, whereas sodium ions inhibit ciliary beat. Therefore, magnesium salts of active compounds will be preferentially included in formulations derived from this invention over sodium ions, with potassium salts also considered as potassium ions also promote cilia beat. Since low overall osmolarity is desired for most applications of this invention, using active compounds where both ions of a salt provide functional benefit is desirable. Therefore magnesium or potassium bicarbonate, magnesium taurate and magnesium hyaluronate are examples of molecules where both ions can be functional in this invention. The present invention is explained in greater detail below.

Specifically, treating and/or managing cystic fibrosis can include any one or more of: improved lung function, improved quality of life, reduced pulmonary exacerbation, reduced the microbial load, reversion of antibiotic susceptibilities of colonizing pathogens, improvement of the gastrointestinal tract and pancreatic function, and treatment of other mucus membranes of the body such as the sinuses.

Lung function can be improved by increasing the patient's forced expiratory volume in one second (FEV1), the forced vital capacity (FVC), and/or whole-lung mucus clearance. Lung function can be measured by spirometry or plethysmography. Lung function can also be assessed by measuring lung volume according to American Thoracic Society standards as described by the American Thoracic Society.

Pulmonary exacerbation is determined by clinical need for IV antibiotics and/or through presence of the following symptoms: change in sputum volume or color, new or increased hemoptysis, increased cough, increased dyspnea, malaise, fatigue or lethargy, a fever, anorexia or weight loss, sinus pain or tenderness, change in sinus discharge, change in findings on physical examination of the chest, decrease in pulmonary function from a previously recorded value, or radiographic change indicative of pulmonary infection.

In a preferred embodiment, the combination drug based on this invention is administered via inhalation. For this route of administration, a drug can contain any of a variety of known aerosol propellants. In addition, a variety of solvents, surfactants, stabilizers (chelating agents and/or antioxidants, inert gases and buffers) can also be included. The formulation can be administered as a dry powder.

An inhaled drug solution can be administered by any aerosolization technique, including but not limited to, standard nebulization. For example, the drug can be delivered using nebulizers or compressors known in the art, such as the Pari LC Plus nebulizer, the Pari Proneb Ultra compressor, Pan LC Star nebulizer, or DeVilbiss nebulizers and compressors. Other types of nebulizers, compressors and aerosolization devices can be used.

Suitable dosages of the present invention can be determined by a physician or qualified medical professional depending on such factors as the nature and/or severity of the illness, route and frequency of administration, the duration of treatment, condition of the patient, the size and age of the patient, and any other relevant factors. One skilled in the art would also know how to monitor the treatment progress in order to determine an effective dose and treatment plan. For example, one skilled in the art could monitor patient spirometry, chest X-rays and CT's, sputum cultures and blood tests. The treatment can be administered as frequently as necessary in order to obtain the desired therapeutic effect of treating the cystic fibrosis or other lung disease or disorder of a mucosal surface.

Frequency of administration will depend, for example, upon the nature of the dosage form used and upon the severity of the condition being treated. For example, if administering in the eye or ear, several drops of the treatment can be administered several times per day. For chronic use, one to two drops of the solution can be administered once to twice daily in certain embodiments. In a non-limiting example, five milliliters of an electrolyzed saline solution is administered once daily, twice daily, or every other day alone or in combination with other therapies as described in more detail below.

In the present invention, an aerosolized drug can be administered in combination with other therapeutic agents administered orally. Such oral therapeutic agents can be administered before, after or concurrently with administration of the inhaled drug. In a non-limiting example, an additional therapeutic agent is administered before, after or concurrently with administration of an electrolyzed saline solution on the same day, or on alternating days. For certain therapeutic agents certain relative time periods of administration can be preferred. In the case of a bronchodilator, it is preferably administered before each inhalation of the invention, however some ingredients within the invention can have bronchodilation effects which are beneficial to the successful penetration of the other ingredients within the formulation(s) derived from the invention. Particular doses of any of these and other additional co-administered therapeutic agents can be determined by a physician or other qualified medical professional depending on factors such as the type of therapeutic agent, the nature and severity of the illness, route and frequency of administration, the duration of treatment, condition of the patient, size and age of the patient, and any other relevant factors.

One of skill in the art would also know how to monitor progress of the treatment in order to determine an effective dose for each treatment as described above. Additional therapeutic agents can also be delivered by a vaporizer, humidifier or fogger. The foregoing descriptions have been supplied merely to illustrate the invention and are not intended to be limiting. Each disclosed aspect and embodiment of the present invention can be considered individually or in combination with any other aspects, embodiments and variations of the invention. In addition, unless otherwise specified, no steps of the methods of the present invention are restricted to any particular order of their performance. Modifications of the disclosed embodiments incorporating the spirit and substance of the present invention can occur to persons skilled in the art and such modifications are within the scope of the present invention.

### Pharmaceutically Acceptable Salts

The term “active agent” as used herein, includes all pharmaceutically acceptable salts of the compound. Pharmaceutically acceptable salts are salts that retain desired biological activities of the parent compound and do not impart undesired toxicological effects. Examples of such salts are (a) salts formed with inorganic acids, for example hydro bromic acid, hydrochloric acid, sulfuric acid, phosphoric acid, nitric acid and the like; and salts formed with organic acids such as, for example, acetic acid, citric acid, oxalic acid, tartaric acid, succinic acid, malic acid, maleic acid, fumaric acid, gluconic acid, ascorbic acid, alginic acid, benzoic acid, tannic acid, palmitic acid, polyglutamic acid, naphthalenesulfonic acid, methanesulfonic acid, p-toluenesulfonic acid, naphthalenedisulfonic acid, polygalacturonic acid, and the like; and (b) salts formed from elemental ions such as chlorine, bromine, magnesium and iodine.

Active agents used to prepare compositions for the present invention can alternatively be in the form of a pharmaceutically acceptable free base form. Because the free base of the compound is less soluble than the salt, free base compositions are employed to provide more sustained release of active agents to the mucosal surface. Active agent present in particulate form which has not yet gone into solution is unavailable to induce a physiological response, but serves as a depot of bioavailable drug which gradually goes into solution.

### Formulations and Administration

The active compounds disclosed herein can be administered to the mucosal surfaces of a patient by any suitable means, including as a dry powder, spray, mist, or droplets of the active compounds in a pharmaceutically acceptable carrier such as distilled water. For example, the active compounds can be selected from the group consisting of magnesium carbonate, potassium bicarbonate, glutathione, a thiocyanate salt, theobromine, theophylline, caffeine, chlorogenic acid, mannitol, xylitol, hyaluronic acid, potassium hyaluronate, TRIS (THAM), thymol, rosmarinic acid, eugenol, boswellic acid, ascorbic acid, ursolic acid, magnesium ascorbate, quercetin, curcumin, luteolin, resveratrol, pterostilbene, apigenin, kaempferol, fisetin, rutin, forskolin, amentoflavone, allicin, vanillin, astaxanthin, retinol acetate, retinol palmitate, N-acetyl cysteine (NAC), N-acetyl lysine (NAL) taurine, magnesium taurate, magnesium sulfate, magnesium ascorbate, guaifenesin, and pycnogenol. These compounds can be prepared as formulations and administered as described in U.S. Pat. No. 5,789,391 to Jacobus.

Similarly, for other mucosal surface diseases, the currently treatments of this invention can be co-administered with therapeutically active drugs, with natural extract compounds and supplements, with vitamins or other compounds that can be advantageously utilized in a combination treatment according to the invention. The disclosed compositions can be administered prior to administration of the known therapeutic, for example at least four hours prior to administration of the known therapeutic. Alternatively, the disclosed compositions can be administered concurrently with the known therapeutic provided there is no adverse interaction with the known therapeutic agent.

The following examples of the present invention in combination described in further detail, but the scope of the invention in any of these examples is not subject to restrictions.

### DETAILED DESCRIPTION OF PREFERRED EMBODIMENTS

Specific examples of active compounds that can be used to carry out the present invention are set forth below.

In one preferred embodiment the invention is administered by nebulization of an aerosol suspension of respirable particles comprised of active compounds, which the subject inhales through the nose and/or mouth. The respirable or particles can be liquid or solid. The quantity of active agents included can be an amount sufficient to achieve dissolved concentrations of active agents on the nasal airway surfaces of the subject of from about 10⁻⁹ to about 10⁻¹ Moles/liter, and more preferably from about 10⁻⁴ to about 10⁻² Moles/liter.

In one embodiment of the invention, the particulate active agent composition can contain both a free base of active agent and a pharmaceutically acceptable salt, to provide both early release of and sustained release of active agents for dissolution into the mucous secretions of the mucosal surface. Such a composition serves to provide both early relief to the patient, and sustained relief over time. Sustained relief, by decreasing daily administrations required, is expected to increase patient compliance with the treatment.

Solid or liquid particulate active agents that are prepared for practicing the present invention can include particles of respirable size: that is, for respirable particles, particles of a size sufficiently small to pass through the mouth and larynx upon inhalation and into the bronchi of the lungs. In general, particles ranging from about 1 to 6 microns in size are respirable. Particles of non-respirable size are greater than about 6 microns in size, up to the size of visible droplets. Thus, for upper airways administration, a particle size in the range of greater than 10 microns can be used to ensure retention in the nasal and sinus cavity if this is desired.

The dosage of active compound will vary depending on the condition being treated and the state of the subject, but generally can be an amount sufficient to achieve dissolved concentrations of active compound on mucosal surfaces of the subject of from about 10⁻⁹ to about 10⁻¹ Moles/liter, and more preferably from 10⁻⁴ to about 5×10⁻² Moles/liter. Depending upon solubility of the particular formulation of active compound administered, the daily dose of the invention can be divided among one or several unit dose administrations. The daily dose by weight in grams per individual active agent within the formulation of the combination drug resulting from this invention can range from about 0.0001 to 3 g of active agent for a human subject, depending upon age and the condition of the subject. A currently preferred unit dose is about 300-450 milligrams of combined total active agents given at a regimen of two to four administrations per day. The dosage can be provided as a prepackaged unit by any suitable means (e.g., encapsulating in a gelatin capsule that can be emptied into a nebulizer cup, or as individual foil packets for use as a nasal irrigation solution).

Pharmaceutical formulations suitable for mucosal surface administration include formulations of solutions, emulsions, suspensions and extracts. See generally, J. Naim, Solutions, Emulsions, Suspensions and Extracts, in Remington: The Science and Practice of Pharmacy, chap. 86 (19^{th} ed. 1995). Pharmaceutical formulations suitable for nasal administration may be prepared as described in U.S. Pat. No. 4,389,393 to Schor; U.S. Pat. No. 5,707,644 to Ilium; U.S. Pat. No. 4,294,829 to Suzuki; and U.S. Pat. No. 4,835,142 to Suzuki.

In the manufacture of a formulation according to the present invention, active agents or the physiologically acceptable salts or free bases thereof are typically admixed with an acceptable carrier. The carrier must, of course, be acceptable in the sense of being compatible with any and all other ingredients in the formulation and must not be deleterious to the patient. The carrier can be a solid or a liquid and is preferably formulated with the compound as a unit-dose formulation. For example, a gelatin capsule can contain from 50% to 99% by weight of the active compound. One or more active compounds are incorporated in the formulations of this invention, which formulations can be prepared by any well-known techniques of pharmacy consisting essentially of admixing the components.

Mists or aerosols of particles comprising the active compounds can be produced by any suitable means, such as by nebulization, or by a simple nasal spray with the active agent in an aqueous pharmaceutically acceptable carrier, such as sterile water. Administration can be by pressure-driven aerosol nebulizer or an ultrasonic nebulizer. See, for example, U.S. Pat. Nos. 4,501,729 and 5,656,256. Suitable formulations for use in a nasal droplet or spray bottle or in nebulizers consist of the active ingredient in a liquid carrier, the active ingredients comprising up to 45% w/w of the formulation, but preferably less than 20% w/w. The carrier is typically water, most preferably sterile pyrogen-free water, or a very dilute aqueous alcoholic solution, preferably made isotonic or modestly hypertonic with body fluids. Optional additives include preservatives if the formulation is not made sterile, for example, thymol, methyl hydroxybenzoate, antioxidants, flavoring agents, volatile oils, buffering agents and surfactants.

Mists or aerosols of the particles comprising the active compounds can be produced with any solid particulate medicament aerosol generator. Aerosol generators for administering solid particulate medicaments to a subject produce particles which are respirable and generate a volume of mist or aerosol containing a predetermined metered dose of a medicament at a rate suitable for human administration. One type of solid particulate aerosol generator is known as an insufflator. Suitable formulations for administration by insufflation include finely ground powders which can be delivered to the patient by means of an insufflator. In the insufflator, the powder (e.g., a metered dose effective to carry out treatments described herein) is contained in capsules or cartridges, typically made of gelatin or plastic, which are either pierced or opened in situ and the powder delivered by air that is drawn through the device upon inhalation or by means of a manually-operated pump. The powder employed in the insufflator consists either solely of the active ingredients or of a powder blend comprising the active ingredients, a suitable powder diluent, such as lactose, and an optional surfactant. The active ingredients typically comprises from 0.1 to 100 w/w of the formulation. A second type of illustrative aerosol generator that can be used with this invention comprises a metered dose inhaler. Metered dose inhalers are pressurized aerosol dispensers, typically containing a suspension or solution formulation of the active ingredients in a liquefied propellant. During use, these devices discharge the therapeutic formulation through a valve adapted to deliver a metered volume, to produce a fine particle spray containing the active ingredients. Suitable propellants include certain chlorofluorocarbon compounds, and mixtures thereof. The formulation can additionally contain one or more co-solvents, for example, ethanol, surfactants (such as oleic acid or sorbitan trioleate), antioxidants and suitable flavoring agents.

Compositions containing respirable dry particles of micronized active agent can be prepared by grinding the dry active agent by mortar and pestle, and then passing the micronized composition through a 400 mesh screen to remove large agglomerates.

The particulate active agent composition can optionally contain a dispersant(s) which serves to facilitate formation of an aerosol. A dispersant can be blended with active agents in any suitable ratio.

One aspect of the current invention is a method for treatment or improvement of pulmonary conditions in cystic fibrosis, asthma, primary ciliary dyskinesia, pulmonary arterial hypertension, idiopathic pulmonary fibrosis, familial pulmonary fibrosis, acute respiratory distress syndrome, persistent pulmonary hypertension of the newborn, chronic obstructive pulmonary disease, acute lung injury and other pulmonary diseases characterized by inflammation and/or infection of mucosal surfaces; by inhalation of a nebulized formulation solution of this invention in a dosage from about 250 mg to about 1500 mg/dose into conducting and central airways, said solution nebulized into an aerosol with a MMAD in the range from about 2 μM to about 10 μM.

Still another aspect of the current invention is a formulation comprising from about 250 to about 1500 mg, preferably about 300-800 mg, per one dose, active components, dissolved in a aqueous solvent wherein said formulation has a pH between 5.5 and 8.5, osmolality between 300 and 1600 mOsm/kg, wherein said formulation is delivered by nebulization in about 1-15 mL, preferably in 3-5 mL, of said formulation, using an electronic, jet or ultrasonic nebulizer optionally equipped with airflow control wherein the resulting aerosol has a MMAD between 3 μM and 10 μM.

Still yet another aspect of the current invention is a dry powder formulation comprising from about 50 to 1000 mg of formulation per one dose, wherein the said formulation is milled, spray dried or precipitated into a fine powder with a MMAD between about 3.0 μM and 10 μM and a substantially wherein said dry powder formulation is used for inhalation administered from one to four times per day.

One preferred formulation for aerosolized formulation comprises formulation dissolved in a minimal volume of about 2.5 to about 5 ml of water. The pH of the solution is adjusted to between about 6.5 and about 7.5. Osmolality of the solution is adjusted to between about 400 and 900 mOsm/kg. The solution is nebulized into an aerosol having mass median aerodynamic diameter (MMAD) between 3 to 8 μm. The formulation solution is aerosolized for substantially into particles having MMAD between 3 and 8 μM.

Another preferred embodiment for aerosolized formulation comprises formulation dissolved in a volume of about 4 to about 6 ml of water. The pH of the solution is adjusted to between about 6.5 and 7.5. Osmolality of the formulation is adjusted to between 450 and 800 mOsm/kg. The solution is nebulized into an aerosol having a mass median aerodynamic diameter (MMAD) between 2 and 6 μM using the electronic nebulizer.

Still another aspect of the present invention is a two-part reconstitution system comprising the formulation as precursor in a concentrated liquid or dry or lyophilized powder form with a diluent stored separately until use. This two-part reconstitution system can be used to tailor the osmolarity of the formulation to the patient's airway tolerance.

Yet another aspect of the current invention is the formulation, containing solution or dry powder, conveniently provided in individual plastic vials for storage at room temperature.

Still another aspect of the current invention is the formulation packaged into gelatin capsules that can be opened to release the powder for reconstitution with solvent or for inhalation in powder form.

The aerosol formulation for nebulization of formulation or another for treatment and tolerance in pulmonary diseases has certain requirements. These requirements include salinity, osmolality, acidity, ion concentration and viscosity of the nebulization solution.

The pH of the drug formulation is an important feature for treatment and treatment tolerance in mucosal diseases. The pH of the formulation must be maintained as close as possible to the neutral pH range for some embodiments, and be elevated in other embodiments, for example for delivery by nebulization into the CF lung. The desired pH range is between 5.5 and 8.5, and is achieved and maintained with biologically acceptable buffers.

When an aerosol to be delivered by nebulization to the lung is either too acidic or too basic, that is, when the pH it is outside of the safe range of pH from 5.5 to 8.5, it can cause bronchospasm in the conducting airways and exacerbate cough. Any aerosol with pH of less than 4.5 or above 8.0 typically induces bronchospasm. Aerosols with the pH between 5.5 and 8.5 may occasionally cause bronchospasm and provoke cough.

Therefore, to avoid development of bronchospasm, cough or inflammation in pulmonary patients, the optimum pH for the amino acid aerosol formulation is determined to be between pH 5.5 and pH 8.5 with preferred pH between pH 7.0 and 8.0.

Therefore the pH range of the formulation arising from this invention is restricted to a range from pH 5.5 to 8.0, and most preferably between pH 7.0 and 8.0.

### Effect of Salinity

Salinity of the formulation resulting from this invention is another important aspect of this invention. Because sweat, tears, saliva, nasal secretions and presumably airway surface liquid of the CF patient are already overly sodium-chloride salty, use of sodium and chloride ions within the formulation will be avoided. Salts of other ions will be used preferentially wherever possible.

Sodium chloride solutions, and sodium ions in particular are known in the art to inhibit activity of cilia. Effective, vigorous movement of cilia are essential to health of respiratory surfaces, and for this reason also use of sodium and chloride ions will be avoided in formulations resulting from this invention.

The chloride ion in the formulation can be substituted with, for example, taurate or ascorbate.

Bronchospasm and cough can be sufficiently controlled and/or suppressed when the salinity and osmolality of the solution are in certain limited ranges. Osmolality of the solution is achieved and can be adjusted with active components of the formulation and by adjusting the volume with water.

The osmolality of an aerosolized solution is another important aspect of the aerosol formulation. Osmolality is directly related to the initiation of bronchoconstriction during inhalation. Bronchospasm and cough are regularly induced by inhalation of solutions with osmolality lower than 100 or higher 1100 mOsm/kg. The optimal osmolality is therefore between 300 and 1100 mOsm/kg.

In one aspect, exception can be made in cases where the increased sputum expectoration (via high osmotic challenge) is desired from the formulation. In such cases the nebulized solution can be brought to 1000 to 1600 mOsm/kg by addition of increased active ingredient per unit volume.

In another aspect of this invention, when the airway hydration is desired, xylitol can be effectively increased within the formulation to raise osmolality up to approximately 1600 mOsm/kg.

For reactive airways, osmolality of the nebulized aerosol solution can optimally, during nebulization, be maintained at osmolality between 300 and 900 mOsm/kg, preferably between 450 and 900 mOsm/kg.

Absence of a permeant anion in nebulized solutions can create a stimulus for cough even under iso-osmolar conditions, and amount of cough is directly proportional to the concentration of permeant anion. Therefore, not only is ion concentration important for airways tolerability of a nebulized solution, but type of ion used must also be considered. Inhalation of a solution with osmolality between ˜230 and ˜600 mOsm/kg induces cough when the permeant ion is less than ˜30 mM Potassium is the most preferred permeant ion because it is not in excess already in CF secretions and does not contribute to impaired ciliary activity like sodium and chloride. A potassium ion concentration between ˜30 and 100 mM is optimal.

In preferred nebulized formulations, the formulation can be formulated as a solution, typically buffered solution or as a salt solution adjusted to desired pH and/or altered according to the needs of the specific formulation. Generally, the liquid formulation does not require salt formulation. For a dry lyophilized form of this invention, the formulation needs to be in a salt form that is reconstituted, prior to aerosolization, with water or a buffered solution. A minimal amount of DMSO or other pharmacologically acceptable solvent can be used to increase solubility of some components of the formulation.

One preferred formulation for aerosolized formulation comprises formulation of all ingredients dissolved in a minimal volume of about 1 to about 5 ml of a pharmaceutically acceptable magnesium or potassium salt. A pH of the solution is adjusted to pH between about 5.0 and about 8.5. Osmolality of the solution is adjusted to between about 300 and 1100 mOsm/kg. The solution is nebulized into an aerosol having a mass median aerodynamic diameter (MMAD) between 3 μM to 10 μM. A suitable nebulizer is the electronic nebulizer. The formulation solution is aerosolized substantially into particles having MMAD between 3 and 10 μM.

Another preferred formulation for aerosolized formulation comprises a buffered formulation of all ingredients dissolved in a volume of about 4 to about 6 ml of normal or diluted pharmaceutically acceptable magnesium or potassium salts. A pH of the solution is adjusted to pH between about 5.5 and 8.5. Osmolality of the solution is adjusted to between 450 and 1100 mOsm/kg. The solution is nebulized into an aerosol having a mass median aerodynamic diameter (MMAD) between 2 and 10 μM using the electronic nebulizer.

### Efficacy of Targeted Delivery of Formulation by Nebulization

A primary requirement of this invention is to efficiently deliver the formulation as active components, to the conducting airways in an efficient and economical way. Delivery of said active components to the lungs is a function of the size distribution of the inhaled aerosol, the delivery system, the volume and the active component content of the particles. Consequently, an initial dose of formulation composition for aerosolization, the actual deposited dose of formulation in the conducting airways, time of nebulization and frequency of the dosing are all important for reaching the best efficacy of the targeted delivery of this invention by nebulization.

### Delivery Time

Delivery time for aerosolization of the entire amount of the active components present in the aerosol plays an important role in the efficacy of the formulation. Given the fact that during nebulization, osmolality of the solution can increase as compared with the pre-nebulization value, it is desirable to deliver the entire volume of the initial aerosol in the shortest possible time in order to maintain osmolality and other parameters of the formulation, as defined above, throughout the nebulization. Change in the osmolality is ultimately translated into change of concentration of the active component in the aerosolizable solution.

Peak increase in osmolality is typically observed between 10 and 15 minutes of nebulization. The rise in osmolality is due to fluid shearing in a high velocity stream of dry gas occurring during nebulization. After generation of primary aerosol droplets during nebulization, solute evaporates from the surface of the aerosol droplets to humidify the air thereby increasing the osmolality in the droplets. Approximately 99% of the droplets then return to the reservoir causing a continuous increase in the concentration of the solute in the liquid remaining in the nebulizer and a continuous increase in the osmolality of the aerosol droplets.

Because of this observable increase in osmolality, the nebulization time can be restricted. With shorter time, there is less measurable change in osmolality and thus reduced concentration effect for the amount of the active component delivered to the lungs.

Proper selection and use of vibrating mesh nebulizers or the other similarly equipped electronic or ultrasonic nebulizer results in shortening of the time required for nebulization, thereby eliminating or negating concentration effects observed with other types of nebulizers. When the time of nebulization is reduced and when the proper nebulizer is selected, there is no change in osmolality and no measurable drug concentration change occurs during nebulization.

Each dose present in the aerosolizable composition solution contains a minimal yet therapeutically effective amount of each active component.

The dose is formulated in the smallest possible volume, and can be adjusted with water to achieve a lower osmolarity as required by the patient and under direction of a qualified healthcare practitioner.

Dose per the whole aerosolizable volume 1-10 mL is 250 to 1500 mg. Dose to be delivered to the lungs is 100 to 600 mg/dose (assuming 40% deposition efficiency). Maximal dose of formulation per day assuming five doses is 7500 mg fill dose/3000 mg deposited.

A total maximum daily administered dose of said active components is therefore between 1250 mg to 7500 mg per day administered in one or more doses of 250 to 1500 mg per one dose. The total maximum deposited daily amount should typically not exceed about 3000 mg per day. The formulation resulting from this invention is administered daily and/or chronically 1 to 5 times per day.

The resulting composition of formulations resulting from this invention is in all cases adjusted such that the aerosolizable solution has an osmolality between 300 and 1600 mOsm/Kg, and pH between 5.5 and 8.5 as the critical parameters.

### Aerosol Volume for Nebulization

The volume of the diluent used for aerosolization of the formulation arising from this invention is also important. Typically, the liquid volume used for nebulization of an inhaled drug is between 1 and 10 mL, preferably between 2 and 6 mL, and most preferably from 2.5-5 mL per single dose. The volume depends on solubility of the formulation in the solute and the dose is adjusted for volume such that the aerosolized solution delivers a therapeutically effective dose of the active components in the most efficient and expeditious way.

### Dosing Regimen

Those of a skill in the art will appreciate that the preferred dosing regimen can be varied depending on the route of administration, symptoms, body weight, health and condition of the patient, and the like, and that the preferred dosing regimen can be readily determined using known techniques.

For desired effect of raising and maintaining the airway surface liquid volume, raising pH of airway surface liquid, increasing function of CFTR, lowering inflammation and providing antimicrobial activity and enhanced cilia activity, the dosing regimen requires either daily administration for a time limited to duration of the disease or conditions in need of treatment, such as in acute lung injury or acute pulmonary hypertension, or daily and/or chronic administration, particularly among chronic diseases, such as cystic fibrosis and primary ciliary dyskinesia.

The formulation resulting from this invention is administered daily and/or chronically 1 to 5 times per day. In the lower of possible delivery efficacies of about 40% from the administered aerosol dose of 250 -1500 mg, the deposited dose in the conducting airways and central lungs would result in from 100 mg to 600 mg of the formulation deposited per one dose. The maximal recommended deposited dose in the target area thus would be from 1800 mg to about and preferably not exceeding 3000 mg of drug deposited per day.

In order to achieve such deposited doses, the nominal (aerosol device fill dose) dose will need to be 250 mg to 1000 mg. The nominal dose will be smallest with devices that have high deposition efficiency, such as vibrating mesh nebulizers coupled with airflow control.

### Efficacy Determination

Efficacy of the targeted delivery of formulation of this invention is measured by the amount of the drug needed to restore innate immune defenses mediated by CFTR transported molecules, to restore extended hydration, to help raise airway surface liquid pH, to inhibit pathogen growth, to reduce inflammation, to directly correct and/or potentiate CFTR function, to thin mucus and/or biofilm and to increase the function of cilia at a mucosal surface. For lung diseases, efficacy is measured in clinical trials primarily by improvements in pulmonary function tests, improved oxygen saturation, improved quality of life and reduced frequency of exacerbations, however other endpoints can be desirable to include.

The amount of exhaled nitric oxide or saliva nitrate can be measured to determine efficacy of the formulation. In addition, forced expiratory volume per one second (FEV1) will be measured via spirometry, and also exacerbation rate, blood O2 saturation, CF quality of life measures (CFQ-R), and exercise capacity will be determined.

### Product Shelf-Life and Storage

Stability of the formulation resulting from this invention is another important issue for efficacious use. If the drug is degraded prior to nebulization, a smaller amount of the drug is delivered to the lungs, thereby reducing the efficacy of the treatment. Moreover, degradation of stored active components may generate degradation products that are poorly tolerated by patients. The dry powder or lyophilized formulation for preparation of the solution for inhalation should have, preferably, at least a one year long shelf life.

The formulation consisting of the active components is prepared aseptically as a lyophilized powder either for dry powder delivery or for reconstitution in sterile water. Alternatively, the formulation can be prepared as a frozen solution, as a liposomal suspension or as microscopic particles. The extended shelf-life of these alternative preparations provide for easy and reliable storage of the formulation resulting from this invention and allows easy reconstitution for use.

In practice, the formulation or another for inhalation suitable for aerosolization can be preferably provided as two separate components, one containing a dry formulation or powder, or a salt thereof, and a second containing an appropriate diluent such as sterile water, as described above. The solution for inhalation is reconstituted immediately prior to aerosolization and administration to the patient. This two component packaging for storage prevents problems connected with long-term stability of the active component in aqueous solvents.

### Combination of the Aerosolized Formulation with Other Therapies

As an alternative strategy, this therapeutic approach for treatment of pulmonary diseases with an aerosolized composition comprising formulation can be advantageously combined and/or augmented with other pulmonary therapies. In particular, the aerosolized formulation can be advantageously combined with a beta-agonist, steroid, anti-inflammatory agent, antibiotic, bronchodilator, mucolytic or another suitable drug.

The formulations of this invention, for example, provide a mechanism to improve the pulmonary condition in cystic fibrosis and can, therefore, be effectively combined with other currently existing and known therapies. Individual therapeutic combinations, doses and specific formulations will depend on the interaction with the other drug(s). The optimization of these combinations is based on the knowledge available in the art.

Safety endpoints for evaluation of this invention are: FEV1, systemic (blood) and urine levels of formulation active ingredients, GI symptoms and other adverse events such as dyspnea, and chest tightness.

Efficacy endpoints for the determination of efficacy of the present invention are: pulmonary function (FEV1), and exhaled nitric oxide (NO). Chest X-rays and CT scans may also be taken. Exploratory endpoints for determination of efficacy are: sputum expectoration and blue dye or saccharin technique of measuring nasal mucociliary clearance.

As described in the following examples, experiments were performed to demonstrate that the formulations resulting from the invention disclosed herein are effective, at least in vitro, to significantly restore CFTR function and reduce inflammation, are safe and well- tolerated wherein administered in vivo, and are effective in treating cystic fibrosis.

### EXAMPLES

### Example 1: Preparation of Composition 1

Components are added to a 1L volumetric flask and brought to a final volume using deionized distilled water. Calculated osmolarity is 842 mOsm/L.

| COMPONENT | Grams per Liter of distilled water |
|---|---|
| Reduced glutathione | 25 |
| TRIS | 20 |
| Taurine | 20 |
| Xylitol | 15 |
| Magnesium bicarbonate | 10 |
| Magnesium ascorbate | 10 |
| Potassium hyaluronate | 3 |
| Apigenin | 0.4 |
| Thymol | 0.2 |

### Example 2: Preparation of Composition 2

Components are added to a 1L volumetric flask and brought to a final volume using deionized distilled water. Calculated osmolarity is 908 mOsm/L.

| COMPONENT | Grams per Liter of distilled water |
|---|---|
| Taurine | 25 |
| Curcumin | 25 |
| Xylitol | 25 |
| TRIS | 15 |
| Reduced glutathione | 15 |
| Magnesium bicarbonate | 15 |
| Magnesium ascorbate | 10 |
| Potassium hyaluronate | 2 |
| Allicin | 1 |
| Naringen | 0.5 |
| Luteolin | 0.25 |
| Thymol | 0.2 |

The present invention is not to be limited in scope by specific embodiments described herein, which are intended as single illustrations of individual aspects of the invention, and functionally equivalent methods and components not provided as examples are within the scope of the invention. Indeed, various modifications of the invention, in addition to those shown and described herein will become apparent to those skilled in the art from the foregoing description and accompanying figures. Such modifications are intended to fall within the scope of the appended claims.

The invention is a composition suitable for the treatment of a mucosal surface in need of such treatment, wherein said composition is prepared as an dry powder or as a solution comprising from about 100 mg to about 1500 mg of total ingredients per dose, with active ingredients selected from the group consisting of molecules known (1) to be actively transported by CFTR (e.g., carbonate/bicarbonate ions, glutathione, thiocyanate and the like), or differentially abundant in CF vs non-CF mucosal secretions (2) and also including molecules intended to restore extended hydration to the mucosal surface (e.g., any alcohol sugar such as mannitol or xylitol, and/or any other pharmaceutically acceptable osmolytes, or hyaluronic acid), (3) and also including molecules intended to balance pH at the mucosal surface such as pharmaceutically acceptable salts of bicarbonate, or TRIS (tris(hydroxymethyl)aminomethane) also known as THAM, or any other pharmaceutically acceptable buffer, and also molecules intended to (4) inhibit pathogen growth at the mucosal surface such as natural plant-derived molecules that are quorum sensing inhibitors for bacteria (thymol, eugenol, quercetin and the like) and/or also natural or synthetic molecules that inhibit pathogen growth by other means such as antimicrobial metals (e.g. silver), and also included in the composition are (5) molecules that reduce inflammation at the mucosal surface (e.g., proanthocyanidins, anthocyanins, procyanidins, catechins, flavones, flavonoids, isoflavones, curcuminoids, stilbenoids, terpenes, carotenoids, rutosides, bithiazoles, pyrazolylthiazoles, benzoquinoliziums, xanthines, benzimidazoles, thiocyanates, isothiocyanates, omega-3 fatty acids and phenolic acids, with examples from this list such as astaxanthin or pycnogenol, resveratrol, pterostilbene, luteolin, quercetin, eicosapentaenoic acid, evodiamine and evodol and also included are (6) molecules that correct and/or potentiate and/or increase (modulate) CFTR function when applied to a mucosal surface, such as certain plant polyphenols (e.g., flavones, flavonoids, isoflavones, curcuminoids, stilbenoids, terpenes, carotenoids, rutosides, bithiazoles, pyrazolylthiazoles, benzoquinoliziums, xanthines, benzimidazoles, thiocyanates, isothiocyanates and the like), and molecules such as forskolin that increase cyclic AMP, thereby activating CFTR, and molecules that are natural phosphodiesterase inhibitors that maintain CFTR function by maintaining cyclic AMP levels such as amentoflavone, and also (7) molecules that have mucolytic activity such bicarbonate salts, and/or thiol containing molecules such as glutathione, or taurine or guaifenesin, or N-acetylcysteine or more preferably N-acetyl lysine, and also (8) molecules that promote mucociliary clearance, which include but are not limited to, compounds that liberate potassium or more preferably magnesium ions in solution.

This invention is set forth in the following claims. Because multiple substrates are transported by CFTR and their functions are complex and interdependent, an effective nebulized therapy for CF must provide restoration and/or compensation for multiple processes that are abnormal at the diseased mucosal surfaces, which are not as completely addressed by prior art.

1. A composition, consisting of combinations of four or more natural and/or synthetic molecules aside from water, that is suitable for the treatment of a diseased mucosal surface, and that fulfill five or more of the following categories and associated function, one or more of which categories and function can be fulfilled by a single molecule:
(1) Molecule(s) that defective CFTR fails to transport, or are otherwise reduced in cystic fibrosis secretions, and which restore mucosal surface innate immune functions
(2) Molecule(s) that serve to restore extended hydration to the affected mucosal surface
(3) Molecule(s) that increase mucosal surface liquid pH
(4) Molecule(s) that inhibit pathogen growth
(5) Molecule(s) that reduce inflammation and/or oxidative stress and/or production of inflammatory mediators
(6) Molecule(s) that directly or indirectly modulate CFTR function
(7) Molecule(s) that provide a mucolytic function
(8) Molecule(s) that increase function of cilia

2. A composition according to claim 1, wherein the compounds are selected from the group consisting of magnesium carbonate, potassium bicarbonate, glutathione, a thiocyanate salt, theobromine, theophylline, caffeine, chlorogenic acid, mannitol, xylitol, hyaluronic acid, potassium hyaluronate, TRIS (THAM), thymol, rosmarinic acid, eugenol, boswellic acid, ascorbic acid, ursolic acid, magnesium ascorbate, quercetin, curcumin, luteolin, resveratrol, pterostilbene, apigenin, kaempferol, fisetin, rutin, forskolin, amentoflavone, allicin, vanillin, astaxanthin, retinol acetate, retinol palmitate, N-acetyl cysteine (NAC), N-acetyl lysine (NAL) taurine, magnesium taurate, magnesium sulfate, magnesium ascorbate, guaifenesin, and pycnogenol.

3. The composition of claim 1 wherein said composition is the aerosolizable solution for inhalation to treat a condition of the lung, wherein said composition is dissolved in about 3 to 10 ml of buffered solution having adjusted pH between about pH 6.5 and pH 9, and osmolality between about 300 and 1400 mOsm.

4. The composition of claim 1 where the mucosal surface disease in need of treatment is selected among: smoker's cough, inflammatory lung disease, pulmonary fibrosis, pulmonary vasculitis, pulmonary sarcoidosis, inflammation and/or infection associated with lung transplantation, acute lung rejection, burn wounds, chemical injury, military wound, pulmonary artery hypertension, bronchitis, sinusitis, asthma, ocular inflammation, ocular infection, dry eye, cystic fibrosis, bacterial infection, fungal infection, parasite infection, viral infection, chronic obstructive pulmonary disease (COPD), sinus infection, bronchiolitis obliterans syndrome (BOS), primary ciliary dyskinesia (PCD), alveolar proteinosis, idiopathic pulmonary fibrosis, familial pulmonary fibrosis, military wounds, burn wounds, eosinophilic pneumonia, eosinophilic bronchitis, acute lung injury, acute respiratory distress syndrome (ARDS), inflammation and/or infection associated with mechanical ventilation, ventilator-associated pneumonia, asbestos-related airway disorder or disease, dust-related airway disorder or disease, silicosis, chemical agent-related airway disease or disorder and any combination thereof.
